(19) United States
(12) Patent Application Publication
(10) Pub. No.: US 2025/0073077 A1
(43) Pub. Date: Mar. 6, 2025

(54) METHOD FOR PROVIDING CONTROL DATA FOR AN OPHTHALMOLOGICAL LASER OF A TREATMENT APPARATUS

(71) Applicant: SCHWIND EYE-TECH-SOLUTIONS GMBH, Kleinostheim (DE)

(72) Inventors: Pascal NAUBEREIT, Laufach (DE); Samuel ARBA MOSQUERA, Aschaffenburg (DE)

(21) Appl. No.: 18/817,613

(22) Filed: Aug. 28, 2024

(30) Foreign Application Priority Data
Aug. 30, 2023 (DE) 10 2023 123 355.6

(51) Int. Cl. A61F 9/008

(52) U.S. Cl. CPC A61F 9/00804; A61F 9/00827; A61F 2009/00844; A61F 2009/00872; A61F 2009/00878

(57) ABSTRACT

The invention relates to a treatment apparatus (10) and to a method for providing control data for an ophthalmological laser (12) of a treatment apparatus (10). The method incudes capturing (S10) at least one eye picture of an eye (16) by a camera device (28); ascertaining (S12) phenomenological structures (24) of the eye (16) from the eye picture by a computing device (18), wherein the computing device (18) determines the phenomenological structures (24) by an image analysis algorithm; ascertaining (S14) treatment positions (26) in the eye for the treatment with the ophthalmological laser (12) depending on the phenomenological structures (24) by the computing device (18); and providing (S16) control data for the treatment apparatus (10) by the computing device (18), which includes the treatment positions (26) in the eye (16) for the treatment.

### FIELD

The invention relates to a method for providing control data for an ophthalmological laser of a treatment apparatus. Furthermore, the invention relates to a treatment apparatus with at least one ophthalmological laser, which is formed to perform the method.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device may for example be formed such that laser pulses effect a photodisruption and/or ablation, in particular a plasma-assisted ablation, in a focus situated within the organic tissue, to remove a tissue, in particular a tissue lenticule, from the cornea.

Therein, a treatment area for treating the cornea is usually set by a user of the treatment apparatus and treatment positions in the treatment area are determined for irradiation with the laser. However, if problems arise in the irradiation of the treatment area, for example the treatment has to be interrupted or aborted, it is often difficult to again adjust the originally planned treatment area and to continue the irradiation.

### SUMMARY

It is the object of the invention to improve a planning of treatment positions, in particular with respect to an inadequately performed pre-treatment, for a treatment with an ophthalmological laser.

This object is solved by the independent claims. Advantageous developments of the invention are disclosed in the examples provided herein, in the following description as well as the figures.

The invention is based on the idea that a positioning of laser pulses in the eye is performed based on phenomenological structures, which may be recognized in the eye by a camera device of the treatment apparatus. In particular, a previously performed treatment, which has for example been inadequately performed, may be recognized by the camera device, in particular already irradiated positions in the eye, and new treatment positions, for example for a post-treatment, may then be adjusted based on the recognized already irradiated positions.

An aspect of the invention relates to a method for providing control data for an ophthalmological laser of a treatment apparatus. The method includes acquiring at least one eye picture of an eye by a camera device, ascertaining phenomenological structures of the eye from the eye picture by a computing device, wherein the computing device determines the phenomenological structures by an image analysis algorithm, ascertaining treatment positions in the eye for the treatment with the ophthalmological laser depending on the phenomenological structures by the computing device, and providing control data for the treatment apparatus by the computing device, which includes the treatment positions in the eye for the treatment.

In other words, an optical eye picture of an eye to be treated may be captured by a camera device, which in particular comprises one or more cameras. The eye picture may subsequently be analyzed by an image analysis algorithm, which is operated by a computing device, for determining phenomenological structures in the eye. Phenomenological structures may for example be structures in an iris, a shape and/or position of the pupil, optionally also in relation to a visual axis, a shape, e.g. a curvature and/or gradient and/or size of the cornea, and/or unnaturally altered areas of the cornea, such as for example an existing, beginning or progressing deformation of the cornea. In particular, the phenomenological structures may also include already treated positions in the cornea, for example inadequately or incompletely processed areas in the cornea, which have already been irradiated with a laser. Herein, the image analysis algorithm may use an edge detection and/or intensity detection to ascertain the structures. Alternatively or additionally, the image recognition algorithm may be trained to preset structures and thus recognize structures or deviations from structures by artificial intelligence.

After ascertaining the phenomenological structures, a planning of treatment positions in the eye may be performed by the computing device, which are to be irradiated by the ophthalmological laser. Therein, the treatment positions may be determined based on the ascertained phenomenological structures. For example, an adjustment and/or positioning of a predetermined treatment profile may be performed, for example a centering in a pupil center and/or a corneal center. Furthermore, unnaturally or pathologically altered areas in the cornea may be recognized as phenomenological structures and a treatment for removing these structures may be positioned and/or treatment positions for completing an inadequate or incomplete pre-treatment may be ascertained to complete the pre-treatment.

Finally, control data may be provided for the treatment apparatus by the computing device, which includes the ascertained treatment positions for the treatment. The treatment apparatus, in particular the ophthalmological laser and/or a beam deflection device, may be controlled by the computing device or a control device by the control data. The control data may include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser may be included in the control data.

The camera device and/or the computing device may belong to the treatment apparatus or be provided separately thereto. The computing device may be an appliance or an appliance component, in particular a computer or processor, which may control the method steps.

By the invention, the advantage arises that a planning of treatment positions may be more accurately performed, whereby a treatment with the treatment apparatus is improved.

The invention also includes embodiments, by which additional advantages arise.

In one embodiment the acquisition of the at least one eye picture is effected of an eye, which is treated with an ophthalmological laser device, ascertaining phenomenological structures of the eye is ascertaining previously treated positions of the eye from the eye picture, wherein the computing device determines the previously treated positions by an image analysis algorithm, ascertaining treatment positions in the eye is ascertaining treatment positions in the eye for the post-treatment with the ophthalmological laser depending on the previously treated positions of the eye, and providing control data is provided for the post-treatment of the eye. This means that the phenomenological structures are previously treated positions, which have for example been generated in an aborted pre- treatment, and new treatment positions are determined based on the ascertained previously treated positions, in particular for a post-treatment of the eye. The previously treated positions, which may in particular be inadequate or incomplete, may for example be recognized from the eye picture due to an opacity or a change of reflection properties by the image recognition algorithm, which may recognize the previously treated positions for example by an edge detection and/or an intensity distribution (threshold value method) and/or due to a comparison to the untreated eye. The laser device, with which the eye has been treated in advance, may be the treatment apparatus or one different therefrom. In particular, the previously treated positions, which may be incomplete, may have been irradiated with an excimer laser and/or a femtosecond laser and for example include an edge incision which does not lead up to the surface, at positions in the cornea in which a treatment abortion has occurred during the treatment or before the treatment and/or the incision was finished, which may for example occur due to a vacuum break of a fixing device and/or a treatment stop of a surgeon. Besides an eye surgical treatment, the new treatment positions may also be made for a laser-induced refractive index change for correcting a previous treatment and/or an accompanying correction of a progressive eye change, in particular presbyopia. By this embodiment, the advantage arises that inadequate or incomplete pre-treatments may be recognized by the camera device and a subsequent treatment for completing or correcting the pre-treatment may be planned thereon, which results in an improvement of the treatment.

In another embodiment, in ascertaining the treatment positions for the post-treatment of the eye, still untreated positions of the eye are determined. This means that a treatment abortion may have occurred in a pre-treatment and thus not the entire treatment area has been irradiated with the laser. By the camera device, the previously irradiated areas may then be ascertained and the remaining areas may be complemented by the newly ascertained treatment positions.

In particular, it is provided that the still untreated positions of the eye are determined from a difference of a preset entire treatment area and the ascertained previously treated positions. The entire treatment area may be preset from predetermined examination data and/or previously planned control data of the laser device. Thus, a complementary area to the previously treated positions or a difference of the entire treatment area to the previously treated positions may be used to determine the untreated positions.

In another embodiment both the previously treated positions and the still untreated positions in the eye are set as the treatment positions, wherein first irradiation parameters are set for the treatment positions of the already treated positions in the control data and second irradiation parameters for the not yet treated positions, wherein the first and second irradiation parameters differ from each other. In other words, the entire treatment area may be again post-treated, wherein other irradiation parameters may be adjusted for already treated positions than for the untreated positions. Herein, an energy and/or a spatial pulse distance may for example be adapted as the irradiation parameters. In particular, it may be provided that already irradiated positions are irradiated with a lower energy deposition than still unirradiated treatment positions. Alternatively, it may also be provided that the entire treatment area, which includes the treatment positions previously irradiated and still to be irradiated, is again irradiated with the same irradiation parameters.

In another embodiment the ophthalmological laser device comprises the camera device, by which the at least one eye picture would be acquired in the pre-treatment, or the previous treatment, of the eye. In other words, the laser device, which differs from the treatment apparatus, may comprise the camera device, by which the eye picture was captured. Thus, the eye picture originates from the previous treatment, wherein it may be transferred to the new treatment with the treatment apparatus. Using the eye picture from the pre-treatment results in the advantage that treated areas are still well visible, in particular an opacity, which decreases over time. The eye picture from the pre-treatment may be scaled by adaptations, for example shifts, rotations and/or scalings, to a coordinate system of the treatment apparatus. In particular, the treatment positions may also be effected based on phenomenological structures of the eye detected in terms of image processing, which are stored in image and/or video data.

In another embodiment the treatment apparatus comprises the camera device and the computing device, by which ascertaining the treatment positions before the post-treatment is performed. Alternatively, an additional diagnostic device may also be provided, which determines the treatment positions and transmits the control data to the treatment apparatus. The advantage that the treatment apparatus comprises both the camera device and the computing device is in that further adaptations to the eye picture, in particular scalings of the eye picture, do not become required and post-treatment may be started directly.

In particular, it is provided that the treatment positions are determined for an incision in the cornea and/or an ablation of corneal tissue and/or a laser-induced refractive index change.

In another embodiment the eye picture is captured by the camera device in a visual and/or infrared spectral range. This means that the camera device may include a light source, which may radiate light in a visual and/or infrared spectral range. Accordingly, the camera device may comprise a camera, which is formed to detect the correspondingly reflected light signal of the light source.

In another embodiment the camera device includes a slit lamp, by which multiple eye pictures with respectively changed slit adjustment are captured, wherein the phenomenological structures of the eye are ascertained from the multiple eye pictures. By a slit lamp, a sharply limited slit-shaped light beam may be directed to the eye to capture sections of the eye, in particular also in a depth direction. Thus, the eye may be scanned by the slit lamp to capture the phenomenological structures, in particular already treated positions of the eye. Herein, the slit adjustment may include a slit width and/or a slit position, which may be adjusted for scanning the eye. Hereby, the advantage arises that the phenomenological structures of the eye may be recognized in an improved manner.

A further aspect of the invention relates to a treatment apparatus with at least one ophthalmological laser for the treatment of a human or animal eye by optical breakdown, in particular photodisruption and/or photoablation, and/or by a laser-induced optical refractive index change, wherein the treatment apparatus includes at least one camera device and a computing device, wherein the treatment apparatus is formed to perform a previously mentioned method. Herein, the same advantages and possibilities of variation as in the method arise. In particular, the treatment apparatus may also include a control device, which is formed to control the laser by the provided control data, wherein the control device may be the computing device.

The method may include at least one additional step, which is executed if and only if an application case or an application situation occurs, which has not been explicitly described here. For example, the step may include the output of an error message and/or the output of a request for inputting a user feedback. Additionally or alternatively, it may be provided that a default setting and/or a predetermined initial state are adjusted.

A further aspect relates to a control device, which is formed to perform the steps of at least one embodiment of the previously described method. Thereto, the control device may comprise a computing unit for electronic data processing such as for example a processor. The computing unit may include at least one microcontroller and/or at least one microprocessor. The computing unit may be configured as an integrated circuit and/or microchip. Furthermore, the control device may include an (electronic) data memory or a storage unit. A program code may be stored on the data memory, by which the steps of the respective embodiment of the respective method are encoded. The program code may include the control data for the respective laser. The program code may be executed by the computing unit, whereby the control device is caused to execute the respective embodiment. The control device may be formed as a control chip or control unit. The control device may for example be encompassed by a computer or computer cluster.

A further aspect relates to a computer program. The computer program includes commands, which for example form a program code. The program code may include at least one control dataset with the respective control data for the respective laser. Upon execution of the program code by a computer or a computer cluster, it is caused to execute the previously described method or at least one embodiment thereof.

A further aspect relates to a computer-readable medium (storage medium), on which the above mentioned computer program and the commands thereof, respectively, are stored. For executing the computer program, a computer or a computer cluster may access the computer-readable medium and read out the content thereof. The storage medium is for example formed as a data memory, in particular at least partially as a volatile or a non-volatile data memory. A non-volatile data memory may be a flash memory and/or an SSD (solid state drive) and/or a hard disk. A volatile data memory may be a RAM (random access memory). For example, the commands may be present as a source code of a programming language and/or as assembler and/or as a binary code.

Further features and advantages of one of the described aspects of the invention may result from the developments of another one of the aspects of the invention. Thus, the features of the embodiments of the invention may be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following may be present in any combination with each other and/or the features of the embodiments. This means, the features of the execution examples may supplement and/or replace the features of the embodiments and vice versa. Thus, embodiments are also to be regarded as encompassed and disclosed by the invention, which are not explicitly shown or explained in the figures, but arise from and may be generated by separated feature combinations from the execution examples and/or embodiments. Thus, embodiments are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic method diagram for providing control data according to an exemplary embodiment.

### DETAILED DESCRIPTION

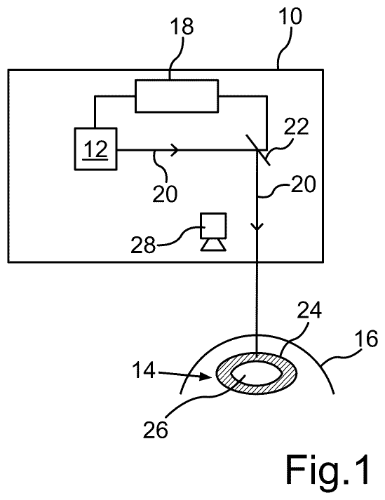

FIG. 1 shows a schematic representation of a treatment apparatus 10 with an ophthalmological laser 12 for treating an eye 16, in particular a cornea of the eye 16, by photodisruption and/or photoablation and/or a laser-induced refractive index change. In this example, for the treatment, an incision surface 14, for example an anterior and/or posterior incision surface for generating a lenticule in the cornea of the eye 16, may be separated from the cornea with the ophthalmological laser 12 for correcting a visual disorder. Treatment positions and/or a geometry of the incision surface 14 may be provided by a computing device 18, which may be formed as a control device of the treatment apparatus 10, in particular in the form of control data, such that the laser 12 emits pulsed laser pulses in a pattern predefined by the control data into the eye 16 to generate the incision surface 14. Alternatively, the computing device 18 may be a computing device 18 external with respect to the treatment apparatus 10.

Furthermore, FIG. 1 shows that the laser beam 20 generated by the laser may be deflected towards the eye 16 into the cornea by a beam deflection device 22 such as e.g. a rotation scanner, to generate the incision surface 14. The beam deflection device 22 may also be controlled by the computing device 18.

The illustrated laser 12 may be a photodisruptive and/or photoablative laser, which is formed to emit laser pulses in a wavelength range between 150 nm and 1400 nm, for example between 700 nm and 1200 nm or between 150 nm and 250 nm, at a respective pulse duration between one femtosecond and 15 nanoseconds, for example between 10 femtoseconds and 10 picoseconds or between 5 nanoseconds and 15 nanoseconds, and a repetition frequency between 500 Hz and 10 kHz, for example between 100 kHz and 100 MHz or between 500 Hz and 1050 Hz. In addition, the computing device 18 optionally comprises a storage device (not illustrated) for at least temporary storage of at least one control dataset, wherein the control dataset or datasets include(s) control data for positioning and/or for focusing individual laser pulses in the cornea of the eye 16.

Furthermore, the treatment apparatus 10 may comprise a camera device 28, which may be formed to acquire eye pictures in a visual and/or infrared spectral range, in particular to capture phenomenological structures in the eye 16.

In a treatment with the treatment apparatus 10 and/or a pre-treatment with a further ophthalmological laser device (not shown), it may occur that the treatment has to be aborted or is inadequate. Thus, an area 24, which includes previously treated positions, and an area 26, which comprises still untreated treatment positions, may for example be present in the incision 14. For example, it may have been planned that the incision surface 14 is generated by a spiral pattern, which leads from the outside to the inside such that the inner area 26 is not irradiated upon abortion of the treatment.

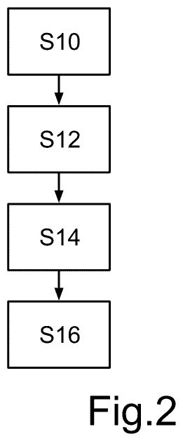

In order to complete such an incomplete treatment or to plan a treatment planning on other phenomenological structures of the eye 16, for example an adjustment of the treatment based on structures of the iris, the method shown in FIG. 2 may be performed.

In FIG. 2, a schematic method diagram for providing control data for the ophthalmological laser 12 of the treatment apparatus 10 is illustrated. By the method, treatment positions in the eye 16 may generally be planned depending on recognized phenomenological structures in the eye, wherein the provision of control data based on the uncompleted incision 14 in the cornea is exemplarily described below, wherein the phenomenological structures may be already treated positions 24 in the eye 16 here.

In a step S10, at least one eye picture of the eye 16 may be captured by the camera device 28, wherein the eye picture includes the previously treated, or already treated positions 24. Alternatively, the camera device 28 may also belong to a laser device, by which the pre-treatment has been performed, such that the eye pictures may be communicated to the treatment apparatus 10, in particular the computing device 18, afterwards. The camera device 28 may acquire the eye picture in a visual and/or infrared spectral range.

In a step S12, the computing device 18 may determine the previously treated, or already treated positions 24 from the eye picture by an image analysis algorithm, for example by an edge detection and/or based on a detection of an intensity distribution. In particular, the camera device 28 may also include a slit lamp (not shown), by which individual areas may be scanned, in order that a recognition of the phenomenological structures (previously treated positions 24) by the image analysis algorithm may be simplified.

In a step S14, the treatment positions 26 for the post-treatment with the laser 12 may be ascertained based on the previously treated positions 24. Thereto, the difference range of the entire incision surface 14 and the previously treated positions 24 may for example be ascertained to determine the still untreated treatment positions 26.

It may also be provided that the entire incision surface 14 is again defined as the treatment area, wherein the already irradiated positions 24 are planned for new irradiation with irradiation parameters different from the still unirradiated irradiation positions 26. Therein, the still unirradiated irradiation positions 26 may in particular be planned with a higher power density than the already irradiated positions 24.

Finally, control data may be provided for the treatment apparatus 10 by the computing device 18 in a step S16, in particular for post-treatment of the eye 16, wherein the control data includes the treatment positions 26. The laser 12 and/or the beam deflection device 22 may then be controlled by the computing device 18 for the irradiation.

Overall, the examples show, how a treatment positioning with support by an image analysis algorithm may be provided.

1. A method for providing control data for an ophthalmological laser of a treatment apparatus, wherein the method comprises the following steps:
capturing at least one eye picture of an eye by a camera device;
ascertaining phenomenological structures of the eye from the at least one eye picture by a computing device, wherein the computing device determines the phenomenological structures by an image analysis algorithm;
ascertaining treatment positions in the eye for a treatment with the ophthalmological laser depending on the phenomenological structures by the computing device; and
providing control data for the treatment apparatus by the computing device, which includes the treatment positions in the eye for the treatment.

2. The method according to claim 1, wherein
capturing the at least one eye picture is effected of an eye which has previously been treated with an ophthalmological laser device;
ascertaining phenomenological structures of the eye includes ascertaining previously treated positions of the eye from the at least one eye picture, wherein the computing device determines the previously treated positions by the image analysis algorithm;
ascertaining treatment positions in the eye includes ascertaining treatment positions in the eye for post-treatment with the ophthalmological laser depending on the previously treated positions of the eye; and
providing control data includes providing control data for the post-treatment of the eye.

3. The method according to claim 2, wherein still untreated positions of the eye are determined in ascertaining the treatment positions for the post-treatment of the eye.

4. The method according to claim 3, wherein the still untreated positions of the eye are determined from a difference of a preset entire treatment area and the ascertained previously treated positions.

5. The method according to claim 4, wherein both the previously treated positions and the still untreated positions in the eye are set as the treatment positions, wherein first irradiation parameters are set for the treatment positions of the previously treated positions in the control data and second irradiation parameters for the still untreated positions, wherein the first and second irradiation parameters differ from each other.

6. The method according to claim 2, wherein the ophthalmological laser device comprises the camera device, by which the at least one eye picture has been acquired in the previous treatment of the eye.

7. The method according to claim 2, wherein the treatment apparatus comprises the camera device and the computing device, by which ascertaining the treatment positions before the post-treatment is performed.

8. The method according to claim 2, wherein the treatment positions are determined for an incision in a cornea and/or an ablation of corneal tissue and/or a laser-induced refractive index change.

9. The method according to claim 1, wherein the at least one eye picture is captured by the camera device in a visual and/or infrared spectral range.

10. The method according to claim 1, wherein the camera device includes a slit lamp, by which multiple eye pictures with respectively changed slit adjustment are captured, wherein the phenomenological structures of the eye are ascertained from the multiple eye pictures.

11. A treatment apparatus with at least one ophthalmological laser for the treatment of a human or animal eye by optical breakdown and/or by a laser-induced optical refractive index change, wherein the treatment apparatus includes at least one camera device and a computing device, wherein the treatment apparatus is configured to perform the method according to claim 1.
